(19) United States
(12) Patent Application Publication
(10) Pub. No.: US 2024/0240268 A1
(43) Pub. Date: Jul. 18, 2024

(54) SAMPLE COLLECTION DEVICES AND METHODS OF USING THE SAME

(71) Applicants: Salus Discovery, LLC, Madison, WI (US); Flambeau Diagnostics, LLC, Monona, WI (US)

(72) Inventors: Jay Warrick, Madison, WI (US); Brianna Mullins, Deforest, WI (US); Patrick McMinn, Madison, WI (US); David Beebe, Monona, WI (US)

(21) Appl. No.: 18/289,354

(22) PCT Filed: May 5, 2022
(86) PCT No.: PCT/US2022/027891

§ 371 Date: Nov. 2, 2023

Related U.S. Application Data
(60) Provisional application No. 63/184,325, filed on May 5, 2021.

(51) Int. Cl. C12Q 1/70; A61B 5/08; A61B 5/097; C12Q 1/6844

(52) U.S. Cl. CPC C12Q 1/701; A61B 5/082; A61B 5/097; C12Q 1/6844

(57) ABSTRACT

Provided herein are sample collection devices and methods of using the same for collecting a sample from a subject. In some aspects, provided herein are wearable sample collection devices. In some embodiments, provided herein are face masks containing a sample collection device and methods of using the same for collection and/or assessment of a sample. In some aspects, provided herein are sample collection devices and methods of using the same for detecting a pathogen in a sample collected from a subject.

### STATEMENT REGARDING RELATED APPLICATIONS

This application claims priority to U.S. Provisional Patent Application No. 63/184,325, filed May 5, 2021, the entire contents of which are incorporated herein by reference for all purposes.

### STATEMENT OF GOVERNMENT INTEREST

This invention was made with government support under RADx contract number 75N92020C00017, awarded by the NIH. The government has certain rights in the invention.

### TECHNICAL FIELD

The invention generally concerns specimen collection for testing.

Provided herein are sample collection devices and methods of using the same for collecting a sample from a subject. In some aspects, provided herein are wearable sample collection devices. In some embodiments, provided herein are face masks containing a sample collection device and methods of using the same for collection and/or assessment of a sample. In some aspects, provided herein are sample collection devices and methods of using the same for detecting a pathogen in a sample collected from a subject.

### BACKGROUND

In the wake of the global COVID-19 pandemic, improved methods for rapid and efficient testing for the presence of pathogens in a sample are of critical importance. In particular, methods for efficient sample collection and subsequent analysis of the sample to determine whether an individual is infected with a pathogen such as a SARS-COV-2 are critical in order to prevent transmission of disease. A key challenge to pathogen detection is extraction/purification of the pathogens (or markers thereof) from biological samples as extraction adds significant time and cost to assays. For this reason, technologies that can either improve sample extraction or even eliminate the step altogether across a wide range of pathogens with pandemic potential are critical.

### BRIEF SUMMARY

The inventions described and claimed herein have many attributes and embodiments including, but not limited to, those set forth or described or referenced in this Brief Summary. It is not intended to be all-inclusive, and the inventions described and claimed herein are not limited to or by the features or embodiments identified in this introduction, which is included for purposes of illustration only and not restriction.

In some aspects, provided herein are sample collection devices. In some embodiments, provided herein is a device for collecting a sample from a subject. In some embodiments, the device comprises a hollow component comprising a first opening permitting entry of particles expelled by the subject into the hollow component and an inner surface for controlling flow of the expelled particles. In some embodiments, the device further comprises a structure connected to the hollow component that limits transmission of particles expelled by the subject to the external environment. In some embodiments, the structure connected to the hollow component is removable. For example, in some embodiments the structure connected to the hollow component comprises a cap.

In some embodiments, the device includes sample collection material comprising a material insert housed on an inner surface of the structure connected to the hollow component, such that the material insert collects particles expelled by the subject. In some embodiments, the material insert comprises a porous material. Any suitable porous material may be used, including natural materials or synthetic materials. For example, any suitable porous polymer may be used. In some embodiments, the material insert comprises cotton, polystyrene, polyurethane, polypropylene, or nylon.

In some embodiments, the inner surface of the hollow component comprises a solid material. Any suitable solid material may be used, including plastics, metals, or ceramics.

In some embodiments, the hollow component is the sample collection material. In some embodiments, at least a portion of the inner surface of the hollow component comprises a porous material, wherein the porous material is the sample collection material. The porous material may permit a portion of air expelled by the subject to exit the device through the porous material. Any suitable porous material may be used, including natural materials or synthetic materials. For example, any suitable porous polymer may be used. In some embodiments, the material insert comprises cotton, polystyrene, polyurethane, polypropylene, or nylon.

In some embodiments, the sample collection material comprises a material insert placed within the hollow component of the device. In some embodiments, the material insert comprises a porous material. Any suitable porous material may be used, including natural materials or synthetic materials. For example, any suitable porous polymer may be used. In some embodiments, the material insert comprises cotton, polystyrene, polyurethane, polypropylene, or nylon.

In some embodiments, the structure connects to the hollow component such that a portion of air expelled by the subject is able to exit the device. In some embodiments, the device further comprises a filter that removes contaminants from the portion of the air exiting the device. In some embodiments, the device further comprises an indicator for determining the amount of sample that has been collected from the subject.

In some aspects, provided herein are systems comprising the devices described herein. In some embodiments, provided herein is a system comprising a device described herein and a face mask. The face mask may be worn by the subject during use of the device, to prevent the spread of particles during use of the device.

The devices described herein may be used in methods of collecting a sample from a subject.

In some aspects, provided herein are methods for collecting a sample from a subject. In some embodiments, the method comprises providing to the subject a device for collecting a sample from a subject. The device may be any suitable device described herein. In some embodiments, the device comprises a hollow component comprising a first opening permitting entry of particles expelled by the subject into the hollow component and an inner surface for controlling flow of the expelled particles. In some embodiments, the device comprises a structure connected to the hollow component that limits transmission of particles expelled by the subject to the external environment. In some embodiments, the device comprises a sample collection material housed on or within the device.

The method further comprises obtaining the sample collection material or particles captured by the sample collection material from the device. For example, obtaining the sample collection material may comprise removing a material insert housed within the hollow component of the device. As another example, obtaining the sample collection material may comprise removing the hollow component of the device. As another example, obtaining the sample collection material may comprise removing a porous material housed on an inner surface of the hollow component of the device. As yet another example, obtaining the sample collection material may comprise removing a material insert housed on an inner surface of the structure connected to the hollow component of the device (e.g. a cap).

The method further comprises detecting one or more pathogens in the sample collection material. In some embodiments, detecting one or more pathogens comprises placing the sample collection material or particles eluted from the sample collection material into a system containing reagents for detection of the one or more pathogens. In some embodiments, the system comprises a container or reaction vessel (e.g. a tube, a test tube, etc.) containing reagents for detection of the one or more pathogens. Any suitable container or reaction vessel may be used. Any suitable reagents may be used. In some embodiments, reagents for detection of the one or more pathogens comprise reagents for a loop mediated isothermal amplification (LAMP)-based assay. In some embodiments, the LAMP-based assay may be a colorimetric or a fluorescent assay. In other embodiments, the reagents for detecting the target comprise reagents for PCR, RT-PCR, qPCR, qtPCR, multiplex PCR, assembly PCR or asymmetric PCR, for example.

In some embodiments, reagents comprise primers for detecting a virus. In some embodiments, reagents comprise primers for detecting a SARS-COV-2 virus, a coronavirus, a rhinovirus, an influenza virus, a respiratory syncytial virus, an adenovirus, a parainfluenza, a human immunodeficiency virus, a human papillomavirus, a rotavirus, a hepatitis virus, a zika virus (e.g. hepatitis A, B, C, D, E), an Ebola virus. In some embodiments, reagents comprise primers for detecting a bacterium or other pathogen. In some embodiments, reagents comprise primers for detecting Mycobacterium tuberculosis, Borrelia burgdorferi, Staphylococcus, Aspergillus, or Streptococcus pyogenes.

In some embodiments, reagents may be retained on a bottom surface of the container or reaction vessel (e.g. test tube) by a temperature-sensitive sealant. In some embodiments, the temperature-sensitive sealant comprises wax or oil.

In some embodiments, methods for detecting one or more pathogens in a sample further comprise inverting the container or reaction vessel (e.g. tube) after placing the sample collection material or particles eluted from the sample collection material into the tube, and incubating the inverted tube at a suitable temperature for a suitable duration to melt the temperature-sensitive sealing and thereby release the reagents for detection of the one or more pathogens. For example, in some embodiments the inverted tube is incubated at a temperature of 50-70° C. for 10-60 minutes. In some embodiments, the method comprises incubating the inverted tube at 65° C. for 25-35 minutes. In some embodiments, the method further comprises cooling the tube in an upright position and detecting one or more pathogens in the tube. In some embodiments, the tube is cooled at a temperature of 0° C.-10° C. for 1-10 minutes.

In some aspects, provided herein are wearable systems for collecting a sample from a subject. In some embodiments, provided herein is a wearable system for collecting a sample from a subject, comprising a face mask and a sample collection device housed within the face mask. The sample collection device may be any suitable sample collection device described herein. In some embodiments, the sample collection device comprises a hollow component operably connected to the mask. In some embodiments, the hollow component comprises a first opening permitting entry of particles expelled by the subject into the hollow component, and a removable collection component that attaches to the hollow component. In some embodiments, the removable collection component captures the sample and prevents transmission of particles expelled by the subject to the external environment. In some embodiments, the removable collection component comprises a cap. In some embodiments, the removable collection component comprises a material insert housed on an inner surface of the component such that particles expelled by the subject enter the hollow component of the sample collection device and collect on the material insert.

In some embodiments, the hollow component is operably connected to the mask by a grommet. In some embodiments, the material insert comprises a porous material. Any suitable porous material may be used, including natural materials or synthetic materials. For example, any suitable porous polymer may be used. In some embodiments, the material insert comprises cotton, polystyrene, polyurethane, polypropylene, or nylon.

In some embodiments, the system further comprises an indicator for determining the amount of sample that has been collected from the subject.

The systems, including wearable systems described herein, find use in methods of collecting a sample from a subject. In some embodiments, provided herein is a method of collecting a sample from a subject. The method comprises providing to a subject a wearable system comprising a face mask and a sample collection device housed within the face mask as described herein, and collecting the sample collection device or a component thereof from the face mask. In some embodiments, the sample collection device comprises a hollow component operably connected to the mask, wherein the hollow component comprises an inner opening exposed to particles expelled by the subject. In some embodiments, the sample collection device comprises a removable cap that attaches to the hollow component, wherein the cap prevents transmission of particles expelled by the subject to the external environment, and wherein the removal cap comprises a material insert housed on an inner surface of the cap, such that particles expelled by the subject enter the hollow component of the sample collection device and collect on the material insert.

In some embodiments, collecting the sample collection device or a portion thereof from the face mask comprises removing the removable cap from the hollow component. The method may further comprise placing the removable cap on a tube containing reagents for detection of one or more pathogens in the sample. Any suitable reagents may be used. In some embodiments, the tube contains reagents for a LAMP-based assay for detecting one or more pathogens in the sample. For example, the LAMP-based assay may be a colorimetric or a fluorescent assay.

In some embodiments, reagents comprise primers for detecting a SARS-COV-2 virus, a coronavirus, a rhinovirus, an influenza virus, a respiratory syncytial virus, an adenovirus, a parainfluenza, a human immunodeficiency virus, a human papillomavirus, a rotavirus, a hepatitis virus (e.g. hepatitis A, B, C, D, E), a zika virus, an Ebola virus. In some embodiments, reagents comprise primers for detecting a bacterium or other pathogen. In some embodiments, reagents comprise primers for detecting Mycobacterium tuberculosis, Borrelia burgdorferi, Staphylococcus, Aspergillus, or Streptococcus pyogenes.

In some embodiments, the reagents are retained on a bottom surface of the tube by a temperature-sensitive sealant. For example, the temperature-sensitive sealant may comprise wax or oil. In some embodiments, the method comprises inverting the tube after placing the removable cap onto the tube, and incubating the inverted tube at a suitable temperature for a suitable duration to melt the temperature-sensitive sealant, thereby releasing the reagents. For example, the tube may be incubated at a temperature of 50-70° C. for 10-60 minutes. In some embodiments, the method comprises incubating the inverted tube at 65° C. for 25-35 minutes. In some embodiments, the method further comprises cooling the tube in an upright position and detecting one or more pathogens in the tube. For example, the tube may be cooled at a temperature of 0° C.-10ºC for 1-10 minutes. In some embodiments, the sample collection device or a portion thereof comprises a sorbent or functionalized or other target-binding material(s) or carrier(s) that can bind to a pathogen target or portion thereof. In some embodiments, the pathogen target is a nucleic acid and the functionalized materials comprise a solid phase or other carrier. In some embodiments, the solid phase or carrier comprises a paramagnetic particle or a functionalized paramagnetic particle. In some embodiments, the target-binding material is specific for a target. In other embodiments, the target-binding material is designed to bind to more than one or multiple targets.

In some embodiments, the mask is a multiplex collection device, i.e. the sample collection device or a portion thereof from the face mask comprises different materials that can bind to and capture different or separate targets (e.g. more than one pathogen target or portion thereof, such as multiple nucleic acid sequences) or different or separate portions of one target.

### BRIEF DESCRIPTION OF THE DRAWINGS

FIG. 1A-1D show an exemplary wearable system comprising a face mask (e.g. a mask) and a sample collection device housed within the mask. The sample collection device comprises a hollow component operably connected to the mask, such that a user wearing the mask will expel particles (e.g. through coughing or breathing) into the hollow component (FIG. 1A). The sample collection device further comprises a cap operably connected to the hollow component. The cap component can be configured such that the particles expelled by the user are blocked from transmission to the external environment by the cap (FIG. 1B). The inner surface of the cap comprises a material insert (e.g. a cotton pad) that collects particles expelled by the user. (FIG. 1C), such that a subject wearing the mask produces particles (including, for example, respiratory droplets) that contact the insert within the cap. The cap may subsequently be removed from the mask and placed on a tube containing reagents for detection of one or more pathogens.

FIG. 2 shows exemplary vessel (tubes) that may be used in connection with a cap from a wearable system as described herein. For example, the tube may contain reagents necessary, useful, or sufficient for detection of one or more pathogens in a sample collected on a cotton pad housed within the cap. The reagents may be on the bottom of a tube (FIG. 2A). The reagents may be retained on the bottom of the tube by a temperature-sensitive sealant (FIG. 2B). After attaching the cap, the tube may be incubated upside-down at a suitable temperature to allow the temperature-sensitive sealant to melt, thus releasing the detection reagents from the bottom of the tube (FIG. 2C). The detection reagents may subsequently contact the sample (e.g. the sample contained within the cotton pad, as shown in FIG. 1C) and the one or more pathogens contained therein can be detected (FIG. 2D).

FIG. 3 shows the results of an exemplary experiment wherein the limit of detection (LOD) was determined using an exemplary system as described herein. The vessel, in this case a tube, contains reagents for a colorimetric nucleic acid amplification assay. The reagents are pink in color in the absence of sufficient nucleic acid (e.g. pathogenic nucleic acid). Sufficient detection of nucleic acid causes the reagents to turn yellow.

FIG. 4 shows the results of an exemplary experiment wherein the limit of detection (LOD) was compared between cotton pads with and without 5 uL saliva dried onto the pad.

FIG. 5 shows an exemplary sample collection device described herein. The device comprises a hollow component comprising a first opening permitting entry of particles expelled by the subject into the hollow component and an inner surface for controlling flow of the expelled particles, a structure connected the hollow component that limits transmission of particles expelled by the subject to the external environment, and a sample collection material housed on or within the device. In this embodiment, the structure connected to the hollow component is a cap, and the sample collection material is a material insert on an inner surface of the cap.

FIG. 6 shows results of an RT-LAMP assay targeting SARS-COV-2 from clinical samples taken from different parts of facemasks worn by infected individuals. The results are displayed as a heatmap with green indicating a lower time to threshold (thus higher viral load), and red indicating no detection of virus. The heatmap is overlayed on top of a diagram of a cartoon mask-wearer and can be used to guide placement of collection surfaces/cap placement.

### Definitions

To facilitate an understanding of the present disclosure, a number of terms and phrases are defined below:

As used herein, the terms “detect”, “detecting”, or “detection” may describe either the general act of discovering or discerning or the specific observation of a detectably labeled composition. The term “detecting” when used in reference to a target refers to detecting either the presence or the absence of the target in the sample. In some embodiments, “detecting” a target in a sample refers to determining that the target is present in the sample. In some embodiments, “detecting” a target in a sample refers to determining that the target is not present in the sample or is not present in sufficient quantities to be detected in the sample.

The terms “mask” or “face mask” as used interchangeably herein refer to a material that covers at least a portion of the face of a user of the mask. For example, a face mask may cover the mouth of a subject. A face mask may cover the nose of a subject. A face mask may cover the mouth and nose of a subject.

As used herein, the term “sample” is used in the broadest sense and is inclusive of many sample types that may be obtained from a subject using a sample collection device as described herein. In some embodiments, samples contain or are suspected of containing a microorganism (e.g. a pathogenic or disease-causing microorganism). In some embodiments, the sample comprises particles. The term “particles” as used herein with reference to a sample is meant to encompass respiratory droplets and aerosols. Accordingly, the sample may contain particles expelled from a subject by breathing, sneezing, and/or coughing. Particles (e.g. respiratory droplets, aerosols) may contain a pathogen or a portion thereof, such as a virus or a bacteria. For instance, particles may contain viral or bacterial nucleic acid, which may be infective to persons other than the subject.

### DETAILED DESCRIPTION

Provided herein are systems and devices for use in collecting a sample from a subject, and methods of using the same. In some aspects, provided herein are devices for collecting a sample from a subject. In some embodiments, the device comprises a hollow component comprising a first opening permitting entry of particles expelled by the subject into the hollow component and an inner surface for controlling flow of the expelled particles. In some embodiments, the hollow component is circular in shape (e.g. a hollow cylinder). The diameter of the first opening may be any suitable diameter. In some embodiments, the diameter of the first opening is a suitable size to easily fit into the mouth and/or nose of a human subject of any age. For example, the diameter of the first opening may be a suitable size to fit into the nose of an infant, adult, or elderly human subject. As another example, the diameter of the first opening may be a suitable size to fit into the mouth of a human subject of any age (e.g. infant, child, adult, or elderly subject). In some embodiments, the device further comprises a structure connected to the hollow component that limits transmission of particles expelled by the subject to the external environment. In some embodiments, the device further comprises a sample collection material housed on or within the device. In some embodiments, the sample collection material is functionalized. For example, the sample collection material may be functionalized with one or more antibodies, aptamers, and the like to facilitate capture of the desired pathogen within a sample.

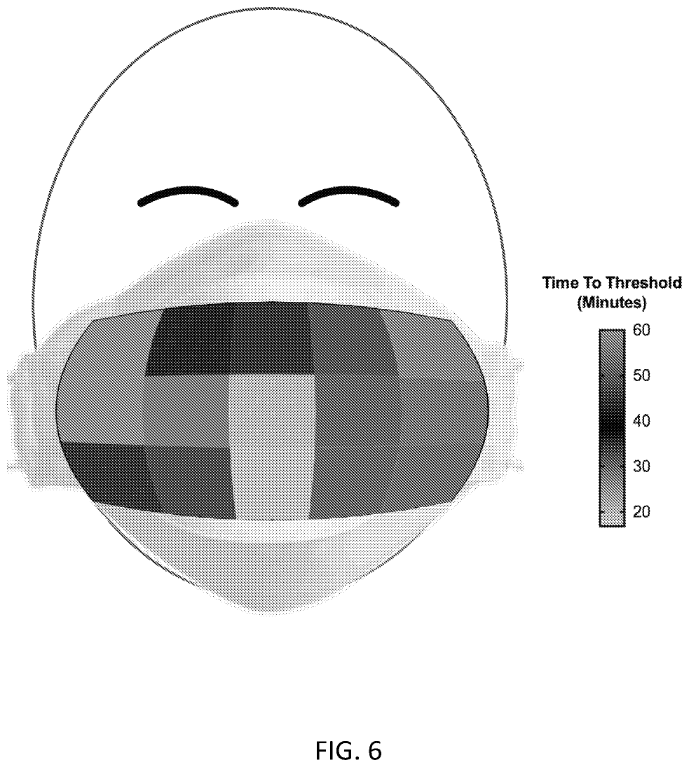

In some embodiments, the structure connected to the hollow component is removable. For example, the structure may be a removable cap (e.g. lid). Such a removable component may be advantageous to facilitate placement of a sample collection material within the device and/or access to samples collected within the device. Cap placement may be guided using the map and results from use of the device as shown FIG. 6, where results of an RT-LAMP assay targeting SARS-COV-2 from clinical samples taken from different parts of facemasks worn by infected individuals are displayed as a heatmap with green indicating a lower time to threshold (thus higher viral load), and red indicating no detection of virus.

In some embodiments, the sample collection material comprises a material insert housed on an inner surface of the structure connected to the hollow component, such that the material insert collects particles expelled by the subject. For example, the sample collection material may comprise a material insert housed on an inner surface of a removable cap. In some embodiments, the sample collection material comprises a material insert placed within the hollow component of the device. For example, a porous material may be placed within the hollow component of the device to serve as the sample collection material. The material insert (e.g. the material insert on an inner surface of the structure connected to the hollow component, or the material insert housed within the hollow component of the device) may comprise any suitable material. In particular embodiments, the material insert comprises an adsorptive porous material. The porous material may be any natural or synthetic material. Suitable porous materials include, for example, cotton (e.g., a cotton pad) nylon, vegetal cellulose, foamed polymers (e.g. polystyrene, polyurethane, polyester, etc.), and the like.

In some embodiments, the inner surface of the hollow component comprises a solid material. In particular embodiments, the entirety of the inner surface of the hollow component is a solid material. Any suitable solid material may be used, including plastics, metals, or ceramics. Such devices may be advantageous in that a majority of the air (e.g. particles contained within the air) expelled by the subject may be directed through the hollow component in a lateral fashion towards a material insert, such as a material insert on an inner surface of the structure connected to the hollow component (e.g. cap) or a material insert housed within the hollow component of the device. Accordingly, such a device acts as a conduit for air expelled by the subject, thereby providing a fast and efficient way to sample an individual's breath. Such a device may be employed for rapid sample acquisition (e.g., at a clinic, or point-of-care, or point-of-need location). Moreover, such a device may enable sufficient pathogens to be captured by the material insert with minimal sample amount provided by the subject. In other words, the subject may only need to provide a single exhale through the mouth or nose in order to provide a sufficient sample to enable subsequent processing of the sample captured by the material insert. Such embodiments may be particularly useful, for example, for detecting viral pathogens in a subject with a high viral titer.

In some embodiments, the hollow component is the sample collection material. For example, in some embodiments, at least a portion of the inner surface of the hollow component comprises a porous material. Such a porous material may serve as the sample collection material. In some embodiments, the entirety of the inner surface of the hollow component is a porous material. In some embodiments, the inner surface is a blend of materials. For example, the inner surface may comprise a solid material (e.g. plastic) with sections of the inner surface comprising a porous material. In such embodiments, the porous material may be removed or particles collected thereupon may be eluted from the porous material to facilitate downstream processing (e.g. pathogen detection) from the material. In some embodiments, the porous material serves as the sample collection material and also permits a portion of air expelled by the subject to exit the device through the porous material. As described above, any suitable porous material may be used including cotton (e.g., a cotton pad) nylon, vegetal cellulose, foamed polymers (e.g. polystyrene, polyurethane, polypropylene, polyester, etc.), and the like. In some embodiments, the porous material allows a portion of air expelled by the subject to exit the device, while the sample is collected on a material insert placed elsewhere (e.g. on the inner surface of a removable cap).

In some embodiments, the structure connects to the hollow component such that a portion of air expelled by the subject is able to exit the device. For example, the structure may connect to the hollow component in such a manner as to leave gaps and/or channels to allow a portion of air to exit the device. In some embodiments, the air expelled by the subject may travel through the hollow component of the device and be directed back towards the subject upon contact with the structure (e.g. cap). For example, as shown in FIG. 5, passage of air expelled by the subject directly through the cap is not feasible. Instead, the cap attaches to the hollow component such that air passes through channels on the perimeter, thereby re-directing the flow of air out of the device and back towards the subject. In this manner, safety of using the device in proximity to others may be improved. Other suitable methods for improving safety (e.g. limiting the spread of particles contained in the breath expelled by the subject) include using filters and/or masks in combination with the device. For example, the device may further comprise a filter that removes contaminants from the air exiting the device. For example, a filter containing suitable pore sizes and/or corrugated folds could be added to the device described herein at a suitable location such that the filter reduces pathogens expelled in the portion of air exiting the device. Alternatively or in addition, the subject may wear a mask over their face while using the device (e.g. while expelling air into the device through their mouth or nose), thereby limiting the spread of pathogens contained in the air exiting the device to bystanders within the external environment.

In some embodiments, the device may further comprise an indicator for determining the amount of sample that has been collected from the subject. For example, an indicator may be a sound indicator. For example, the subject may provide a sample to the device by placing the device into their mouth and expelling air while simultaneously producing sound with their vocal cords (e.g. vocal phonation, similar to playing a kazoo). The duration and relative intensity of the sound provided in doing so may be recorded, thereby ensuring that the person has provided a sufficient sample to be subsequently used in methods for pathogen detection. Similarly, a siren may be used that makes a whizzing sound or other suitable pitch related to the amount of air expelled into the device. Analysis of the sound could provide an indication of the amount of breath sampled. Other suitable indicators may be color change indicators, such as indicators that change color in response to heat and/or moisture expelled through the breath of a subject. For example, carbon dioxide from breath expelled by a subject using the device could be used to cause change of a colorimetric pH indicator in/on the device (e.g., color indicating the extent of breath sampled). Likewise, an indicator that requires moisture to change color may also be used, or a temperature sensitive indicator that responds to the warmth of breath expelled by the subject may be used.

Effective sampling of pathogens and controlling airflow in the devices described herein can also be significantly impacted (positively or negatively) by the process of condensation of moisture from the breath onto surfaces or into porous materials of the device. For example, saturating a porous material with moisture can prevent breath from passing through the material. This could reduce the ability of a portion of the subject's breath to exit the device, such as through a porous filter material. However, saturating a porous material with moisture can also promote capture of pathogens onto the moist surface, such as how mucus aids in the capture of pathogens in the nasal passage. Therefore, the variations on the geometry of the devices described herein may be employed in order to enhance or prevent condensation of moisture from the breath onto one or more components of the device. Such variations may depend on the ability of the subject to effectively expel air. For example, elderly subjects or subjects with compromised lung function may benefit from attempts to prevent the porous materials within the device from becoming saturated with moisture. For example, methods to improve dissipation of heat from the device will maintain a cooler sample collection material, thereby reducing the chances of the material becoming more impervious to breath. Accordingly, for such subjects it may be desirable to cool the device, such as by refrigeration, prior to use. Alternatively or in addition, it may be desirable to soak the device with an evaporative substance, thus cooling the materials within the device through evaporative processes.

Alternatively, in some subjects (e.g. subjects with no difficulty expelling air even when difficult to do so) it may be desirable to improve the chances of saturating the porous materials (e.g. the sample collection material) with moisture in order to improve pathogen capture. For use in such subjects, the device may be heated immediately prior to use or the sample may be obtained from the subject in a humid environment.

Variations in air flow (e.g. tortuosity) may also be employed to promote interaction of different air streamlines and airborne droplets and particles with components of the devices described herein. For example, as the size scale of an air conduit decreases, flow of air becomes more laminar and is often described with the Reynolds number. In laminar flow, streamlines of the fluid (e.g., air) faithfully follow the conduit, making it difficult for the walls of the conduit to interact with particles flowing along central streamlines. However, with curves in the flow, particles or droplets that differ in density are pushed towards the wall, less dense components of the fluid moving toward the inside of the turn while denser components move to the outside of the turn. Likewise, tortuous paths can result in chaotic mixing, even in laminar flow regimes, allowing more streamlines to come into closer proximity to the conduit walls. Therefore, tortuosity may be utilized as a means to better sample each breath of air. For example, in a device as depicted in FIG. 5, forcing the air back toward the user will encourage aerosolized droplets to contact and thus be captured by the capture material. Similarly, the pore size and connectivity of a porous material used in a perfusive device can dramatically enhance tortuosity of the air-flow path to promote sampling of aerosolized droplets from each breath. Typically, increased tortuosity is associated with increased resistance to air-flow. Given that users will likely prefer a device that is easier to breathe through, tortuosity should be balanced with air-resistance. However, increased cross-sectional area to flow can be used to reduce resistance (e.g. a greater diameter of the first opening of the hollow component of the device), but would also increase the surface area of material over which the sample is captured. Thus, concentration of the captured biological sample per unit area would be diminished. Thus, these major parameters should be balanced for each application (e.g. selected based upon the needs of the subject providing the sample). For example, otherwise healthy asymptomatic adults will generally be able to accept a higher flow resistance compared to a baby or seriously ill individual and will be expected to require different strategies or optimal parameters for sampling.

In some embodiments, the device provided herein may be incorporated into a system for sample collection. For example, the system may comprise a face mask to be used in conjunction with the device. As described above, a face mask may be used to increase safety of the device by limiting the amount of potential pathogens expelled into the external environment during use of the device. Such devices and systems described herein find use in methods of collecting a sample from a subject. Moreover, such devices and systems find use in methods for collecting a sample and subsequently detecting one or more pathogens present in the sample. Accordingly, the devices and systems described herein may be designed for facile integration into systems for pathogen detection.

In some aspects, provided herein are wearable systems comprising a face mask and a sample collection device housed within the face mask. In some embodiments, the mask covers at least the mouth of the user. In some embodiments, the mask covers at least the mouth and the nostrils of the user. The mask may be affixed to the face of the user by any suitable means, including affixing straps around the head, ears, jawline, neck, etc. The mask may comprise a component to ensure a snug fit along the ridge of the nose of the users, such as a flexible wire. The mask may comprise any suitable material. The mask may comprise a woven or a non-woven material. For example, the mask may be a natural fabric such as cotton, linen, hemp, silk, cashmere, wool, jute, bamboo, mohair, leather, and the like. Alternatively, the mask may comprise a synthetic fabric such as polyester, nylon, rayon, spandex, acrylic, polymers, copolymers (e.g. Lycra, Spandex), and the like. The mask may comprise a blend of fabrics. In some embodiments, the mask may be a surgical mask. A surgical mask refers to a mask made of nonwoven fabric created using a melt blowing process. In some embodiments, a surgical mask comprises multiple layers made of a melt-blown polymer (e.g. polypropylene) placed between non-woven fabric.

The sample collection device may be housed within the face mask. In some embodiments, the sample collection device comprises a hollow component operably connected to the mask. In some embodiments, the hollow component comprises a first opening permitting entry of particles expelled by the subject into the hollow component. The sample collection device further comprises a removable collection component that attaches to the hollow component, wherein the removable collection component captures the sample and prevents transmission of particles expelled by the subject to the external environment. In some embodiments, the removable collection component comprises a removable cap that attaches to the hollow component, such that the cap prevents transmission of particles expelled by the subject to the external environment. In some embodiments, the removable cap comprises a material insert housed on an inner surface of the removable cap, such that particles expelled by the subject collect on the material insert. The material insert may comprise any suitable material. In some embodiments, the material insert comprises an adsorptive porous material. Suitable porous materials include, for example, cotton (e.g., a cotton pad) nylon, vegetal cellulose, hydrocellulose, foamed polymers (e.g. polystyrene, polyurethane, polyester, etc.), and the like. In some embodiments, the material insert is functionalized. For example, the material insert may be functionalized with one or more antibodies, aptamers, and the like to facilitate capture of the desired pathogen within a sample.

The hollow component may be any suitable size and shape. In some embodiments, the hollow component is a hollow cylinder, such that a circular cap or other collection component can easily be used in conjunction with the hollow component to prevent transmission of particles expelled by the subject to the external environment. In some embodiments, the hollow component is a hollow cylinder with a length of less than 5 cm such that placement of the hollow cylinder within the mask does not alter the comfort and fit of the mask itself for the user. For example, the hollow component may be a hollow cylinder with a length of less than 5 cm, less than 4 cm, less than 3 cm, less than 2 cm, or less than 1 cm. The hollow component may be of any suitable diameter to facilitate entry of air containing particles into the hollow component and enabling sufficient quantities of particles (e.g. pathogens) to collect on the material insert. The hollow component may be operably connected to the mask by any suitable means. In some embodiments, the hollow component is operably connected to the mask by a grommet.

In some embodiments, the removable collection component (e.g. cap) is positioned between two layers of the mask such that it is not visible from the exterior side of the mask. In some embodiments, either an interior surface or an exterior surface of the mask contains an openable panel or other access mechanism to permit removal of the collection component after sample collection is completed. In some such embodiments, the collection component is removed by cutting or tearing an outer or inner surface of the mask.

The removable collection component can be any structure that provides a sufficient construction and volume to permit collection of the sample. While a cap is used to illustrate the invention, and provides an efficient structure for subsequent analysis steps, the invention is not limited to the use of a cap. In some embodiments, the collection component is a substantially liquid impermeable or impermeable housing with an opening to receive sample. While a wide variety of volumes may be used, the collection component is typically sized to be large enough to collect a sufficient sample for subsequent analysis but small enough to conveniently be positioned on or in a mask.

In some embodiments, where the mask is made of a sufficiently porous material, no hollow component is required. In such embodiments, the removable collection component is positioned in a region of the mask to collect sufficient sample that passes through a porous layer of the mask. In such embodiments, a portion of the porous material of the mask may be removed and utilized in downstream sample analysis (e.g. detection of pathogens captured on the porous material).

In a wearable embodiment, such as a mask as described herein, there are also potential methods to indicate how much sample has been acquired. For example, carbon dioxide from breath expelled by a subject wearing the mask could be used to cause change of a colorimetric pH indicator in/on the fabric (e.g., color indicating the extent of breath sampled). Likewise, an indicator that requires moisture to change color may also be used, or a temperature sensitive indicator that responds to the warmth of breath expelled by the subject may be used.

The devices and systems described herein find use in methods for collecting a sample from a subject. The sample may comprise particles expelled from the subject. The method may comprise providing to the subject a system or a device as described herein. In some embodiments, the method comprises providing to the subject a device comprising a hollow component comprising a first opening permitting entry of particles expelled by the subject into the hollow component and an inner surface for controlling flow of the expelled particles, a structure connected the hollow component that limits transmission of particles expelled by the subject to the external environment, and a sample collection material housed on or within the device. The method may further comprise obtaining the sample collection material or particles captured by the sample collection material from the device.

In some embodiments, the method for collecting a sample from the subject comprises providing to the subject a wearable system as described herein (e.g. a wearable system comprising a face mask and a sample collection device housed within the face mask) and collecting the sample collection device or a component thereof from the face mask.

In some embodiments, providing to the subject a device as described herein may comprise placing the device into the mouth or nose of the subject and instructing the subject to breathe into the device or simply waiting for the subject to passively breathe into the device (e.g. such as waiting for an infant to breathe into the device). The methods further comprise obtaining the sample collection material from the device after the subject has expelled sufficient air (e.g. breath) into the device. In some embodiments, the subject may provide a single breath, and or a single breath while phonating (e.g. like playing a kazoo) into the device.

For the wearable sample collection systems described herein, providing the system to the subject may comprise placing the face mask on the subject and/or instructing the subject to place the face mask upon themselves. While the subject is wearing the mask the subject may cough, sneeze, breathe, etc. into the mask. Accordingly, particles expelled by the subject enter into the sample collection device. The subject may wear the mask for a suitable duration of time to allow a sufficient number of particles to enter the sample collection device. In some embodiments, the subject may wear the mask for about 1 minute. In some embodiments, the subject may purposefully cough into the mask to provide a sample. In some embodiments, the subject may wear the mask for more than 1 minute. For example, the subject may wear the mask for at least 1 minute, at least 5 minutes, at least 10 minutes, at least 20 minutes, at least 30 minutes, at least 40 minutes, at least 50 minutes, at least 1 hour, at least 2 hours, at least 3 hours, at least 4 hours, at least 5 hours, or more than 5 hours (e.g. a work day of 8 hours, 10 hours, 12 hours, etc.). After a sufficient amount of time has passed with the subject wearing the mask, the sample collection device or a portion thereof (e.g. the cap) may be removed from the system and used for subsequent analysis of the sample. For example, the cap may be removed from the system and placed on a tube or other vessel containing reagents for pre-detection (e.g. sample preparation, storage, or shipment) or detection of one or more pathogens in the sample.

In some aspects, provided herein are methods for detecting one or more pathogens in a sample. The methods comprise providing to the subject a device or system as described herein. In some embodiments, the methods comprise performing a method for collecting a sample from a subject as described herein, followed by subsequent processing steps to detect one or more pathogens in the sample.

In some embodiments, the methods comprise providing to the subject a device comprising a hollow component comprising a first opening permitting entry of particles expelled by the subject into the hollow component and an inner surface for controlling flow of the expelled particles, a structure connected the hollow component that limits transmission of particles expelled by the subject to the external environment, and a sample collection material housed on or within the device. The methods further comprise obtaining the sample collection material or particles captured by the sample collection material from the device, and placing the sample collection material or particles eluted from the sample collection material into a system containing reagents for detection of the one or more pathogens. For example, the sample collection material may be a material insert placed within the hollow component, or housed on an inner surface of the structure connected to the hollow component (e.g. cap). The material insert may be removed from the device and placed into a system containing reagents for detection of one or more pathogens. As another example, the sample collection material may be a porous material comprising a portion of the inner surface of the hollow component of the device. The porous material may be cut out or otherwise removed from the hollow component and placed into a system containing reagents for detection of one or more pathogens. As yet another example, the particles captured by the sample collection material may be eluted from the material (e.g. eluted with a suitable buffer or other liquid). For example, a volume of elution buffer added to a device, and the device may be centrifuged to pass the buffer through the porous capture material and into a suitable collection device. The liquid (e.g. buffer) containing the eluted particles may be placed into a system containing reagents for detection of the one or more pathogens in the sample (e.g. a testing component).

In some embodiments, the methods comprise providing to a subject a wearable system comprising a face mask and a sample collection device housed within the face mask. In some embodiments, the collection device comprises a hollow component operably connected to the mask. The hollow component comprises an inner opening exposed to particles expelled by the subject, and a removable collection component (e.g. cap) that attaches to the hollow component. In some embodiments, the removable collection component prevents transmission of particles expelled by the subject to the external environment. In some embodiments, an inner surface of the removable cap comprises a material insert. For example, the material insert may be a cotton insert (e.g. a cotton pad). Accordingly, particles expelled by the subject (e.g. by coughing, sneezing, breathing) will enter the hollow component and collect on the material insert contained within a collection component. In some embodiments, the hollow component is part of the collection component.

In some embodiments, the sample collection device or a portion thereof from the face mask comprises sorbents or other target-binding materials or carriers, including functionalized target-binding materials or carriers, that can bind to a pathogen target or portion thereof. In some embodiments, the pathogen target is a nucleic acid and the sorbent is a porous organosilicate sorbent (with or without a stabilizing reagent(s)). In other embodiments, the pathogen target is a nucleic acid and the functionalized materials comprise a solid phase or other carrier. In some embodiments, the solid phase or carrier comprises a paramagnetic particle or a functionalized paramagnetic particle.

In some embodiments, the target-binding material is specific for a target. In other embodiments, the target-binding material is designed to bind to more than one or multiple targets.

In some embodiments, the functionalized solid phase or carrier (e.g., functionalized paramagnetic particle) comprises one or more antibodies, antigen-binding fragments (e.g., F(ab′)2, Fab, Fab′, Fv, etc., generated form the variable region of IgG and IgM, for example, which may vary in size, valency and Fc content), single chain variable fragments (scFV) recombinant antibody fragments (rAbFs), aptamers, peptides and peptidomimetics, natural and chemically modified antisense oligonucleotides, or other suitable agents to assist with capture of a target.

In some embodiments, the mask is a multiplex collection device, i.e. the sample collection device or a portion thereof from the face mask comprises different materials that can bind to and capture different or separate targets (e.g. more than one pathogen target or portion thereof, such as multiple nucleic acid sequences) or different or separate portions of one target. In some embodiments, the capture materials bind to one or more portions of one or more viruses. In some embodiments, the target is a virus(es) is/are any pathogen, including those referred to herein which additionally include a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In other embodiments, the virus is a Poliovirus, a Norwalk virus, a Yellow fever virus, a West Nile virus, a Hepatitis C virus, a Dengue fever virus, a Rubella virus, a Ross River virus, a Sindbis virus, a Chikungunya virus, a Borna disease virus, a Marburg virus, a Measles virus, a Mumps virus, a Nipah virus, a Hendra virus, a Newcastle disease virus, a Rabies virus, a Lassa virus, a Hantavirus, and/or a Crimean-Congo hemorrhagic fever virus.

In some embodiments, the removable collection component, or the material insert housed within the removable collection component, is transferred to a system for detecting a target in the sample (e.g. a testing component). In some embodiments, the target is a protein (e.g. antibody), whole cell, or a nucleic acid (e.g. DNA, RNA). In some embodiments, the target is a metabolite, a carbohydrate, a glycopeptide, or a lipid. In some embodiments, the target is a pathogen. In such embodiments, the sample collection component is transferred to a comprising reagents for detection of pathogens in the sample (e.g. a testing component). For example, the collection component (or material insert) may be transferred to a testing component comprising a tube housing reagents for detection of the target (e.g. pathogen) in the sample.

Any of a wide range of systems containing reagents for detection of one or more pathogens may be used including, but not limited to, tubes, test tubes, wells or multi-well plates, lateral flow devices, microfluidic cards, capillary tubes, and the like.

The system containing reagents for detection of the target (e.g. one or more pathogens) in the sample (e.g. testing component) may contain any suitable reagents for pre-detection or detection of one or more pathogens in the sample. For sample preparation, the testing component may contain one or more lysis reagents. For sample storage or shipment, the testing component may contain one or more stabilization buffers, proteases, or the like. In particular embodiments, the testing component contains reagents for a colorimetric assay for detecting one or more pathogens. Such embodiments allow for a facile visualization of whether or not the sample contains the one or more pathogens of interest. In some embodiments, the testing component contains reagents for a colorimetric detection of one or more nucleic acid target molecules following nucleic acid amplification (PCR, isothermal amplification, etc.). In embodiments wherein the nucleic acid is RNA, the tube or other suitable container may contain reagents for a reverse transcription. In some embodiments, LAMP amplification is employed. LAMP assays or RT-LAMP assays provide rapid reaction times, one-tube processing, and easy visualization of results without the need for expensive equipment or additional materials. In general, LAMP reactions include a DNA polymerase with strong strand displacement activity and tolerance for elevated temperatures and up to six DNA oligonucleotides of a certain architecture. RT-LAMP reactions additionally include a reverse transcriptase. Samples with potential template molecules are added to the reaction and incubated for 20 to 60 min at a constant temperature (e g. 65° C.). The oligonucleotides act as primers for the reverse transcriptase, and additional oligonucleotides for the DNA polymerase are designed so the DNA products loop back at their ends. These, in turn, serve as self-priming templates for the DNA polymerase. In the presence of a few RNA template molecules, a chain reaction is set in motion, which then runs until the added reagents (in particular, the deoxynucleotide triphosphates) are used up.

In some embodiments, the reagents for a colorimetric LAMP assay (or colorimetric RT-LAMP assay) further include an indicator, which permits evaluation of a color change in the sample in the presence of sufficient pathogenic nucleic acid (e g, the nucleic acid which the LAMP reagents are designed to detect). Suitable indicators include pH-sensitive indicators and metal-sensitive indicators. In some embodiments, pH-sensitive indicators (e.g phenol red) may be used, due to their easy visualization with the naked eye. In some embodiments, the testing component comprises reagents for a fluorescent LAMP or a fluorescent RT-LAMP assay.

In some embodiments, the reagents comprise oligonucleotides (e.g. primers) designed for detection of bacterial nucleic acid. In some embodiments, the reagents comprise oligonucleotides designed for detection of viral nucleic acid (e.g. RNA). In some embodiments, the reagents comprise oligonucleotides designed for an infection selected from SARS-COV2, coronavirus, rhinovirus, influenza, respiratory syncytial virus, adenovirus, parainfluenza, human immunodeficiency virus, human papillomavirus, rotavirus, hepatitis C virus, zika virus, Ebola virus, tuberculosis, Borrelia burgdorferi, staphylococcus, aspergillus, and Streptococcus pyogenes. For example, the reagents may comprise oligonucleotides designed for detection of a viral upper respiratory infection selected from SARS-COV-2, SARS, a coronavirus, rhinovirus, influenza, or respiratory syncytial virus. In some embodiments, the reagents comprise oligonucleotides for detection of SARS-COV-2 RNA.

In some embodiments, the reagents are retained toward, at or on a bottom surface of the vessel (e.g. a tube) by a substance to hold them in place (e.g. a sealant). Any type of mechanical or chemical device or construct may be used for this purpose.

In some embodiments, the tube or other vessel comprises a temperature-sensitive sealant. For example, the sealant may be solid at room temperature and change to liquid (e.g. melt) at elevated temperatures, thereby releasing the reagents from the bottom of the tube and allowing contact with the sample. Suitable temperature-sensitive sealants include, for example, waxes or oils that are solid at room temperature. For example, the temperature-sensitive sealant may comprise paraffin wax or coconut oil.

In some embodiments, the methods further comprise inverting the vessel (e.g. tube) (after placing the sample collection material on the tube) and incubating the inverted tube at a suitable temperature to allow the temperature-sensitive sealant to liquefy (e.g. melt). For example, the sample collection material may be a removable cap containing a material insert housed on the inner surface of the cap, and the cap itself may be removed from the device or wearable system and placed on a tube. The tube may subsequently be inverted, such the reagents retained on the bottom of the tube may contact the pathogens contained within the collection material. The temperature and duration of incubation may vary based upon the temperature-sensitive sealant used. In some embodiments, the inverted tube may be incubated at 50-70° C. For example, the inverted tube may be incubated at 50° C., 55° C., 60° C., 65° C., or 70° C. The inverted tube may be incubated for any suitable duration of time. In some embodiments, the inverted tube may be incubated for 10-60 minutes. For example, the inverted tube may be incubated for 10 minutes, 15 minutes, 20 minutes, 25 minutes, 30 minutes, 35 minutes, 40 minutes, 45 minutes, 50 minutes, 55 minutes, or 60 minutes to allow the temperature-sensitive sealant to melt. The reagents contained therein will thus be released and will drop down onto the sample collection material (e.g. cap), which contains the sample. Subsequent contact between the sample and the reagents will allow detection of one or more pathogens in the sample.

In some embodiments, the methods comprise cooling the tube in an upright position prior to detecting one or more pathogens in the sample. The tube may be cooled at any suitable temperature for any suitable duration of time. In some embodiments, a colorimetric assay starts to display results (e.g. a color change will occur or will not occur) during the cooling period. For example, the tube may be cooled at a temperature of 0° C.-10° C. for 1-10 minutes.

In some aspects, provided herein are kits. For example, a wearable system as described herein may be packaged into a kit. As another example, a device as described herein may be packaged into a kit, which may additionally comprise a mask for use in combination with the device to prevent spread of pathogens during use of the device. The kit may further comprise a container (e.g. tube(s)) containing reagents for detection of one or more pathogens. For example, the kit may contain tubes that pair with the removable cap of the sample collection device, such that the kit provides a means for collecting a sample and subsequent detection of one or more pathogens in the sample. In some embodiments, the tubes may contain reagents for a colorimetric assay (e.g. LAMP assay) such that the kit provides a facile means to visually detect one or more pathogens in the sample without additional reagents or the use of expensive equipment. Kits may further comprise appropriate controls and/or detection or indicator reagents. Kits may contain instructions for how to use the kit (e.g. instructions for how to wear the mask, ensure the mask fits properly, instructions for appropriate removal of the sample collection device or a portion thereof (e.g. the cap), instructions for how to perform the detection assay (e.g. colorimetric LAMP assay), etc. Instructions may be provided in written form or available in a digital format (e.g. website link, compact disk, and the like).

The wearable systems, methods, and kits described herein find use in a variety of settings wherein sample collection and subsequent evaluation are critical for preventing unwanted transmission of disease. For example, schools and workplaces may find use of the systems described herein. For example, children at school or employees at a workplace may wear a mask as described herein for a portion or the entirety of the day. Wearing the mask itself may prevent transmission of pathogens, such as pathogens contained in particles expelled by the subject, from the subject to others in the vicinity. At a suitable time point (e.g. at the end of the work-day, end of the school-day, etc.) the subject (or a different individual) may remove the cap from the sample collection device and place the cap upon a suitable tube for detection of pathogens in the sample contained within the cap (e.g. on a material insert on an inner surface of the cap). The tube may be processed appropriately to determine whether the subject is infected with a pathogen or whether the subject is healthy. For example, within an hour, a colorimetric LAMP (or colorimetric RT-LAMP) assay would reveal whether the individual wearing the mask was infected with a pathogen. Therefore, use of a system as described herein would inform employers, schools, and the like whether individuals should return to work/school the following day, or whether they need to initiate appropriate medical treatment procedures for the infection, self-quarantining, etc.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

### EXAMPLES

### Example 1

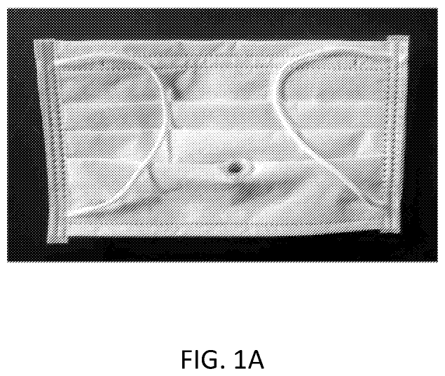

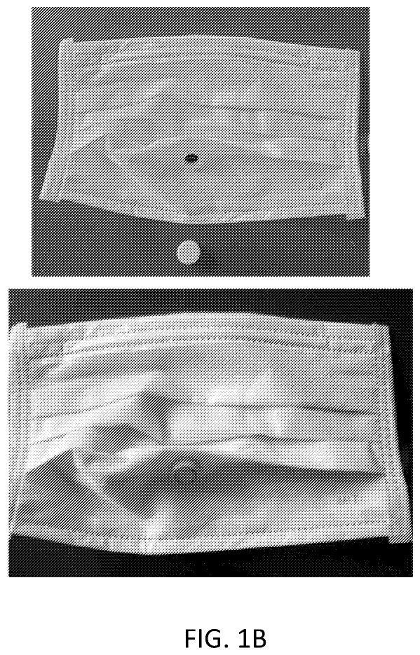

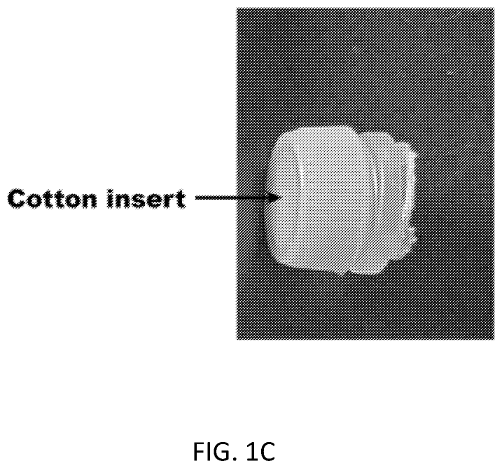

This example provides an exemplary wearable system as described herein. The system comprises a mask containing a sample collection device housed therein. In this example, the mask is a surgical mask. The sample collection device comprises a hollow component that operably connects to the mask, such that a user wearing the mask will expel particles (e.g. through coughing or breathing) into the hollow component (FIG. 1A). The sample collection device further comprises a cap that operably connects to the hollow component, such that the particles expelled by the user will be blocked from transmission to the external environment by the cap (FIG. 1B). The inner surface of the cap comprises a material insert (e.g. a cotton pad) that collects particles expelled by the user. (FIG. 1C), such that a subject wearing the mask will produce droplets that contact the cotton insert within the cap. The cap may subsequently be removed from the mask and placed on a tube containing reagents for detection of one or more pathogens.

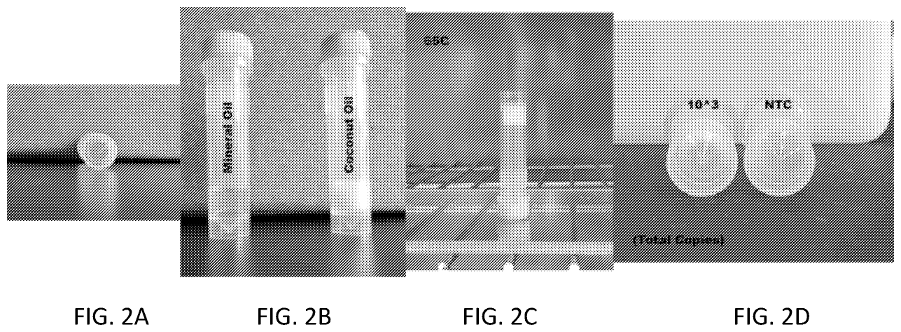

FIG. 2 shows exemplary containers, in this case, tubes, that may be used in connection with a cap from a wearable system as described herein. For example, the tube may contain reagents necessary for detection of one or more pathogens in a sample collected on a cotton pad housed within the cap. The reagents may be on the bottom of a tube (FIG. 2A). The reagents may be retained on the bottom of the tube by a temperature-sensitive sealant (FIG. 2B). After attaching the cap, the tube may be incubated upside-down at a suitable temperature to allow the temperature-sensitive sealant to melt, thus releasing the detection reagents from the bottom of the tube (FIG. 2C). The detection reagents may subsequently contact the sample (e.g. the sample contained within the cotton pad, as shown in FIG. 1C) and the one or more pathogens contained therein can be detected (FIG. 2D).

### Example 2

This example demonstrates the basic mechanism of detaching the cap from the sample collection device and subsequently using the cap in combination with a tube containing reagents for a LAMP assay. For this example, the inner surface of the cap contains a cotton pad. The cotton pad was exposed to a known number of viral copies of nucleic acid. In particular, the pad was exposed to 10\hat{ }3, 10\hat{ }2, 10\hat{ }1, 10\hat{ }0, 10\hat{ }-1, or no copies of the viral RNA. The tubes contain reagents for a colorimetric LAMP assay. The reagents are pink in color in the absence of sufficient nucleic acid. The reagents turn yellow in color in the presence of sufficient nucleic acid.

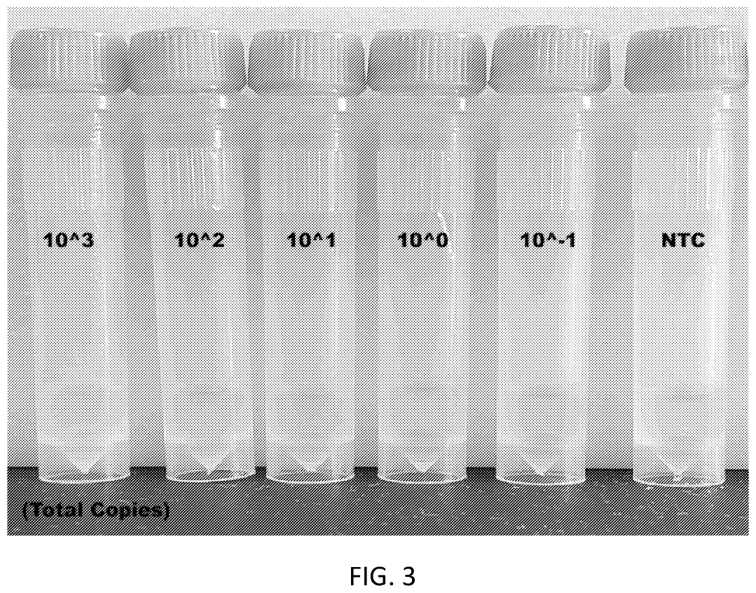

The caps (containing cotton pads exposed to the known number of viral copies of nucleic acid) were placed on the tubes, tubes were inverted and incubated at 65° C. for about 30 minutes. After 30 minutes, tubes were removed from the oven and placed upright at 4° C. for about 5 minutes. Results are shown in FIG. 3. As shown in FIG. 3., the limit of detection (LOD) was determined to be 10\hat{ }1 viral copies.

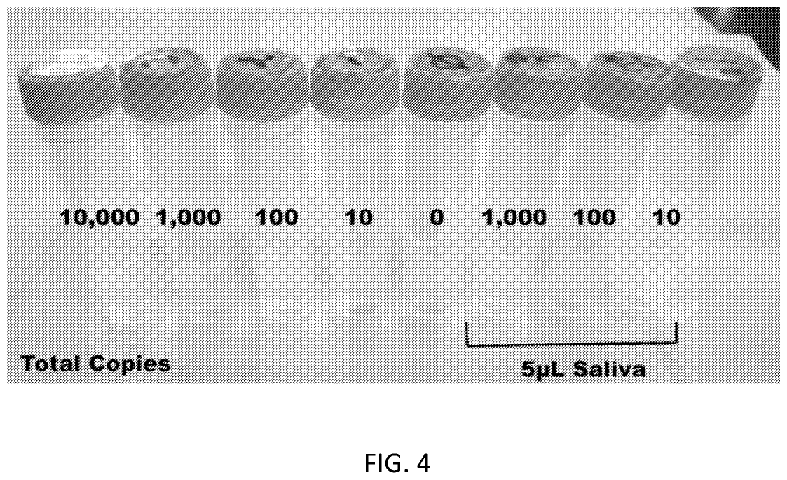

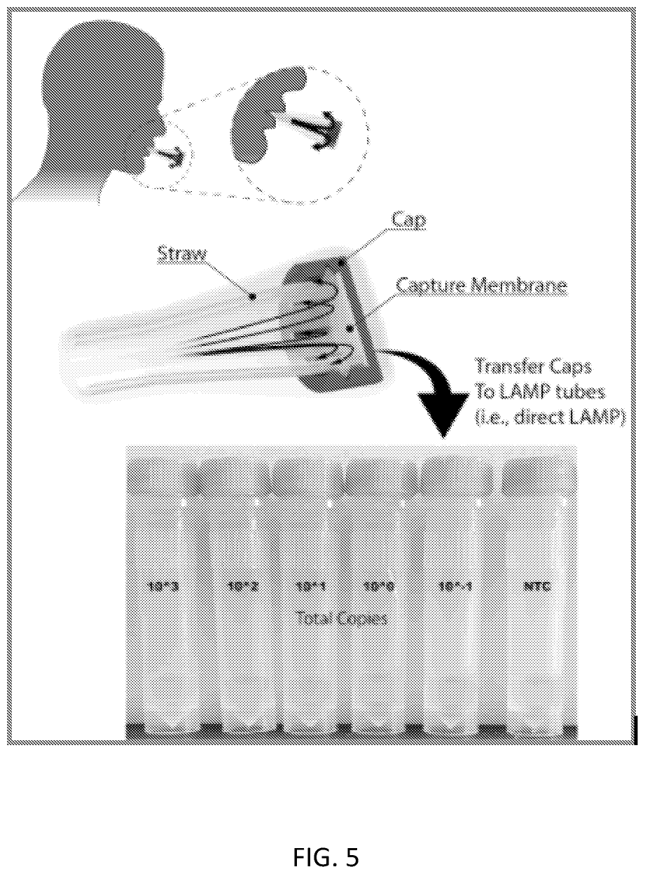

To determine whether dried saliva would impact the LOD, an additional experiment was conducted wherein 5 uL of saliva was dried onto the cotton pads prior to exposing the pads to the viral RNA. The steps of placing the caps on the tubes, inverted incubation, and cooling were performed as described above. Results are shown in FIG. 4. As shown in the figure, the limit of detection (LOD) was determined to be 100 viral copies in pads with and without the dried saliva present. These results show that dried saliva does not lower the LOD, and therefore does not impact the ability of the methods and devices described herein to be used to detect pathogens in a sample.

The results herein demonstrate that if 100 viral copies is the LOD, the cap (e.g. material insert) would need to collect about 10 uL of aerosolized saliva/breath from a subject with a viral load of 10\hat{ }4 copies/mL, or about 1 uL of aerosolized saliva/breath from a subject with a viral load of 10\hat{ }5 copies/mL. The average adult male breathes 16.67 mL of liquid out every hour (which equates to roughly 277 uL/minute). The results presented herein therefore demonstrate that the system described herein presents a feasible methodology to collect sufficient sample from a subject wearing the mask for a minimal amount of time, in some instances for one minute or less.

### Example 3

FIG. 5 shows an exemplary conduit-style sample collection device described herein. The device comprises a hollow component comprising a first opening permitting entry of particles expelled by the subject into the hollow component and an inner surface for controlling flow of the expelled particles, a structure connected the hollow component that limits transmission of particles expelled by the subject to the external environment, and a sample collection material housed on or within the device. In this embodiment, the structure connected to the hollow component is a cap, and the sample collection material is a material insert on an inner surface of the cap.

What is claimed is:

1. A device for collecting a sample from a subject, the device comprising:
a. a hollow component comprising a first opening permitting entry of particles expelled by the subject into the hollow component and an inner surface for controlling flow of the expelled particles,
b. a structure connected to the hollow component that limits transmission of particles expelled by the subject to the external environment, and
c. a sample collection material housed on or within the device.

2. The device of claim 1, wherein the structure connected to the hollow component is removable.

3. The device of claim 2, wherein the structure comprises a cap.

4. The device of any of the preceding claims, wherein the sample collection material comprises a material insert housed on an inner surface of the structure connected to the hollow component, such that the material insert collects particles expelled by the subject.

5. The device of claim 4, wherein the material insert comprises a porous material.

6. The device of claim 5, wherein the material insert comprises cotton, polystyrene, polyurethane, polypropylene, or nylon.

7. The device of any one of the preceding claims, wherein the inner surface of the hollow component comprises a solid material.

8. The device of claim 7, wherein the solid material comprises plastic, metal, or ceramic.

9. The device of claim 1, wherein at least a portion of the inner surface of the hollow component comprises a porous material, wherein the porous material is the sample collection material.

10. The device of claim 9, wherein the porous material permits a portion of air expelled by the subject to exit the device through the porous material.

11. The device of claim 9 or 10, wherein the porous material comprises cotton, polystyrene, polyurethane, polypropylene, or nylon.

12. The device of claim 1, wherein the sample collection material comprises a material insert placed within the hollow component of the device.

13. The device of claim 12, wherein the material insert comprises a porous material selected from cotton, polystyrene, polyurethane, polypropylene, or nylon.

14. The device of any of the preceding claims, wherein the structure connects to the hollow component such that a portion of air expelled by the subject is able to exit the device.

15. The device of claim 14, further comprising a filter that removes contaminants from the portion of the air exiting the device.

16. The device of any of the preceding claims, further comprising an indicator for determining the amount of sample that has been collected from the subject.

17. A system comprising the device of any of the preceding claims and a face mask.

18. The device of any one of claims 1-16 or the system of claim 17 for use in a method of collecting a sample from a subject.

19. A method of collecting a sample from a subject, comprising:
a. providing to the subject a device for collecting a sample from a subject, the device comprising:
i. a hollow component comprising a first opening permitting entry of particles expelled by the subject into the hollow component and an inner surface for controlling flow of the expelled particles,
ii. a structure connected the hollow component that limits transmission of particles expelled by the subject to the external environment, and
iii. a sample collection material housed on or within the device; and
b. obtaining the sample collection material or particles captured by the sample collection material from the device.

20. The method of claim 20, wherein the structure connected to the hollow component is removable.

21. The method of claim 20, wherein the sample collection material comprises a material insert housed on an inner surface of the removable structure such that the material insert collects particles expelled by the subject.

22. The method of claim 21, wherein the material insert comprises a porous material.

23. The method of claim 22, wherein the material insert comprises cotton, polystyrene, polyurethane, polypropylene, or nylon.

24. The method of any one of claims 19-23, wherein the inner surface of the hollow component comprises a solid material.

25. The method of claim 24, wherein the solid material comprises plastic, metal, or ceramic.

26. The method of claim 19, wherein at least a portion of the inner surface of the hollow component comprises a porous material, wherein the porous material is the sample collection material.

27. The method of claim 26, wherein the porous material permits a portion of air expelled by the subject to exit the device through the porous material.

28. The method of claim 26 or 27, wherein the porous material comprises cotton, polystyrene, polyurethane, polypropylene, or nylon.

29. The method of claim 19, wherein the sample collection material comprises a material insert placed within the hollow component of the device.

30. The method of claim 29, wherein the material insert comprises a porous material selected from cotton, polystyrene, polyurethane, polypropylene, or nylon.

31. The method of any of the preceding claims, wherein the structure connects to the hollow component such that a portion of air expelled by the subject is able to exit the device.

32. The method of claim 31, wherein the device further comprises a filter that removes contaminants from the portion of the air exiting the device.

33. The method of any of the preceding claims, wherein the device further comprises an indicator for determining the amount of sample that has been collected from the subject.

34. The method of any of the preceding claims, further comprising detecting one or more pathogens in the sample collection material.

35. The method of claim 34, wherein detecting one or more pathogens comprises placing the sample collection material or particles eluted from the sample collection material into a system containing reagents for detection of the one or more pathogens.

36. The method of claim 35, wherein the system comprises a test vessel containing reagents for detection of the one or more pathogens.

37. The method of claim 35 or 36, wherein the reagents for detection of the one or more pathogens comprise reagents for a loop mediated isothermal amplification (LAMP)-based assay.

38. The method of claim 37, wherein the LAMP-based assay is a colorimetric or a fluorescent assay.

39. The method of any one of claims 36-38, wherein the reagents comprise primers for detecting a SARS-COV-2, a coronavirus, a rhinovirus, an influenza virus, a respiratory syncytial virus, an adenovirus, a parainfluenza virus, a human immunodeficiency virus, a human papillomavirus, a rotavirus, a hepatitis B, C or D virus, zika virus, Ebola virus, tuberculosis, Borrelia burgdorferi, staphylococcus, aspergillus, or Streptococcus pyogenes.

40. The method of any one of claims 36-39, wherein the reagents are retained within the test vessel by a temperature-sensitive sealant.

41. The method of claim 40, wherein the temperature-sensitive sealant comprises wax or oil.

42. The method of claim 40 or 41, further comprising inverting the test vessel after placing the sample collection material or particles eluted from the sample collection material into the tube, and incubating the inverted tube at a temperature of 50-70° C. for 10-60 minutes.

43. The method of claim 42, comprising incubating the inverted test vessel at 65° C. for 25-35 minutes.

44. The method of claim 42 or 43, further comprising cooling the test vessel in an upright position and detecting one or more pathogens in the tube, optionally wherein the test vessel is cooled at a temperature of 0° C.-10° C. for 1-10 minutes.

45. The method of any of claims 40-44, wherein the test vessel is a tube.

46. A wearable system for collecting a sample from a subject, the system comprising:
a. a face mask; and
b. a sample collection device housed within the face mask.

47. The system of claim 46, wherein the sample collection device comprises:
a. a hollow component operably connected to the mask, wherein the hollow component comprises a first opening permitting entry of particles expelled by the subject into the hollow component, and
b. a removable collection component that attaches to the hollow component, wherein the removable collection component captures the sample and prevents transmission of particles expelled by the subject to the external environment.

48. The system of claim 47, wherein the removable collection component comprises a cap, and wherein the cap comprises a material insert housed on an inner surface of the cap such that particles expelled by the subject enter the hollow component of the sample collection device and collect on the material insert.

49. The system of any one of claims 46-48, wherein the hollow component is operably connected to the mask by a grommet.

50. The system of claim 48 or 49, wherein the material insert comprises a porous material.

51. The system of claim 50, wherein the porous material comprises cotton, polystyrene, polyurethane, polypropylene, or nylon.

52. The system of any one of claims 46-51, further comprising an indicator for determining the amount of sample that has been collected from the subject.

53. The system of any one of claims 46-52 for use in a method of collecting a sample from a subject.

54. A method of collecting a sample from a subject, comprising:
a. Providing to a subject a wearable system comprising a face mask and a sample collection device housed within the face mask; and
b. Collecting the sample collection device or a component thereof from the face mask.

55. The method of claim 54, wherein the sample collection device comprises:
a. a hollow component operably connected to the mask, wherein the hollow component comprises an inner opening exposed to particles expelled by the subject, and
b. a removable cap that attaches to the hollow component, wherein the cap prevents transmission of particles expelled by the subject to the external environment, and wherein the removal cap comprises a material insert housed on an inner surface of the cap, such that particles expelled by the subject enter the hollow component of the sample collection device and collect on the material insert.

56. The method of claim 54, wherein collecting the sample collection device or a portion thereof from the face mask comprises removing the removable cap from the hollow component.

57. The method of claim 56, further comprising placing the removable cap on a tube containing reagents for detection of one or more pathogens in the sample.

58. The method of claim 57, wherein the tube contains reagents for a LAMP-based assay for detecting one or more pathogens in the sample.

59. The method of claim 58, wherein the LAMP-based assay is a colorimetric or a fluorescent assay.

60. The method of any one of claims 57-59, wherein the reagents comprise primers for detecting SARS-COV2, coronavirus, rhinovirus, influenza, respiratory syncytial virus, adenovirus, parainfluenza, human immunodeficiency virus, human papillomavirus, rotavirus, hepatitis C virus, zika virus, Ebola virus, tuberculosis, Borrelia burgdorferi, staphylococcus, aspergillus, or Streptococcus pyogenes.

61. The method of any one of claims 57-60, wherein the reagents are retained on a bottom surface of the tube by a temperature-sensitive sealant.

62. The method of claim 61, wherein the temperature-sensitive sealant comprises wax, or oil.

63. The method of claim 61 or 62, further comprising inverting the tube after placing the removable cap onto the tube, and incubating the inverted tube at a temperature of 50-70° C. for 10-60 minutes,

64. The method of claim 63, comprising incubating the inverted tube at 65° C. for 25-35 minutes.

65. The method of claim 63 or 64, further comprising cooling the tube in an upright position and detecting one or more pathogens in the tube.

66. The method of claim 65, wherein the tube is cooled at a temperature of 0° C.-10° C. for 1-10 minutes.

67. A device according to any of claim 1-16 or 18, further comprising at least one target-binding carrier.

68. A device according to claim 67, wherein the target-binding carrier is functionalized.

69. A device according to claim 68, wherein the functionalized target-binding carrier is a paramagnetic particle.

70. A method according to any of claims 19-45, wherein the device for collecting a sample from a subject further comprises at least one target-binding carrier.

71. A method according to any of claims 54-66, wherein the wearable system further comprises at least one target-binding carrier.

72. A method according to any of claim 70 or 71, wherein the target-binding carrier is functionalized.

73. A method according to claim 72, wherein the functionalized target-binding carrier is a paramagnetic particle.

74. A wearable system according to any of claim 17 or 46-53, wherein the sample collection device further comprises at least one target-binding carrier.

75. A wearable system according to claim 74, wherein the target-binding carrier is functionalized, optionally, a functionalized paramagnetic particle.
